(19) United States
(12) Patent Application Publication
(10) Pub. No.: US 2020/0009163 A1
(43) Pub. Date: Jan. 9, 2020

(54) POLYPHOSPHATES AS INHIBITORS OF CALCIUM CRYSTALLIZATION

(71) Applicant: UNIVERSITY OF HOUSTON SYSTEM, Houston, TX (US)

(72) Inventors: Jeffrey D. Rimer, Houston, TX (US); Bryan G. Alamani, Houston, TX (US)

(73) Assignee: UNIVERSITY OF HOUSTON SYSTEM, Houston, TX (US)

(21) Appl. No.: 16/490,959

(22) PCT Filed: Mar. 6, 2018
(86) PCT No.: PCT/US18/21108

§ 371 Date: Sep. 4, 2019

Related U.S. Application Data
(60) Provisional application No. 62/467,659, filed on Mar. 6, 2017.

(51) Int. Cl. A61K 31/6615; A61K 33/42; A61P 13/04; A61P 3/14

(52) U.S. Cl. CPC A61K 31/6615; A61P 3/14; A61P 13/04; A61K 33/42

(57) ABSTRACT

Embodiments described herein generally relate to compositions and methods requiring these compositions that may act to reduce the incidence and/or reoccurrence of kidney stones and/or other pathological calcification diseases, symptoms, or conditions. The compositions include a polyphosphate material, which may be method of treating pathological calcification. In some embodiments, the polyphosphate material included in the composition to be administered may be a linear tripolyphosphate material or a hexametaphosp hate material.

This application claims priority to U.S. Provisional Patent Application No. 62/467,659, entitled “Polyphosphates as Inhibitors of Calcium Crystallization,” filed Mar. 6, 2017, the entire content of which is hereby incorporated by reference.

### FEDERALLY SPONSORED RESEARCH

This invention was made with government support under National Science Foundation, Award#1207441. The government has certain rights in the invention.

### BACKGROUND

This disclosure relates generally to inhibiting mineral crystallization.

Various diseases involving pathological calcification are known. Kidney stones are one example of pathological calcification. Crystallized calcium oxalate is a common constituent of many types of kidney stones and it is thus considered that saturation (attendant to crystallization) of calcium oxalate compounds within the kidneys is likely a precondition to the formation of these types of kidney stones. While calcium oxalate stones are a common type of kidney stone, calcium phosphate (such as brushite) stones are also prevalent.

While various treatments for kidney stones exist and may be effective, they do not always prevent post-treatment reoccurrence of kidney stones. Some existing kidney stone treatments are physically invasive and thus carry significant risks to the patient. Drug-based treatments relying on compounds such as hydrochlorothiazide, sodium phosphate, and potassium citrate are available, but effectiveness (and side effects) may vary patient-to-patient. Some compounds, such as citrate and hydroxycitrate, which act to dissolve calcium oxalate crystals that have formed within the body, are known, but new treatments for pathological calcification could be beneficial to some patients.

### SUMMARY

In one embodiment, a composition for inhibiting pathological calcification comprises a polyphosphate material and a pharmaceutically acceptable carrier. Polyphosphate material may be a polyphosphate, a polyphosphate derivate, or combinations including a polyphosphate and a polyphosphate derivative.

In another embodiment, a method of treating pathological calcification comprises administering a composition to a patient, the composition including a polyphosphate material and a pharmaceutically acceptable carrier. In some examples, the composition may be administered in a therapeutically effective amount to the patient. Pathological calcification includes, without limitation, abnormal biomineralization associated with kidney stones, hypercalciuria, gout, and atherosclerosis.

In still another embodiment, a method of controlling pathological calcification in a patient comprises administering a composition including at least one of a linear tripolyphosphate material and a hexametaphosphate material. A linear tripolyphosphate material may be a linear tripolyphosphate, a derivative of linear tripolyphosphate, or combinations including a linear tripolyphosphate and a derivative of linear tripolyphosphate. Similarly, a hexametaphosphate material may be a hexametaphosphate, a derivative of hexametaphosphate, or combinations including a hexametaphosphate and a derivative of hexametaphosphate.

### BRIEF DESCRIPTION OF THE DRAWINGS

FIG. 1A depicts a linear polyphosphate.

FIG. 1B depicts a functionalized/derivative form of a linear polyphosphate.

FIG. 1C depicts a hexametaphosphate.

FIG. 1D depicts a functionalized/derivative form of a hexametaphosphate.

FIG. 1E depicts a branched polyphosphate.

FIG. 1F depicts a functionalized/derivative form of a branched polyphosphate.

FIG. 1G depicts a functionalized/derivative form of a linear polyphosphate.

FIG. 2A depicts a non-exhaustive collection of possible functional groups that can be used as or incorporated into “R” groups on various polyphosphate materials in accordance with preferred embodiments described herein.

FIG. 2B depicts additional exemplary functional groups that can be used as or incorporated into “R” groups on various polyphosphate materials in accordance with preferred embodiments described herein.

FIG. 2C depicts additional exemplary functional groups that can be used as or incorporated into “R” groups on various polyphosphate materials in accordance with preferred embodiments described herein.

FIG. 3 depicts experimental results obtained using various polyphosphate materials to inhibit crystallization of calcium oxalate from aqueous solution.

FIG. 4A depicts optical microscope images showing results of calcium oxalate crystallization from a control solution and solutions including a linear polyphosphate material Na₅P₃O₁₀ at concentrations of 5 μM and 15 μM.

FIG. 4B depicts effects of Na₅P₃O₁₀ concentrations on calcium oxalate crystal frequency.

FIG. 5A depicts optical microscope images showing results of calcium oxalate crystallization from a control solution and solutions including a cyclic polyphosphate material (NaPO₃)₆ at concentrations of 0.3 μM and 0.7 μM.

FIG. 5B depicts effects of (NaPO₃)₆ concentrations on calcium oxalate crystal frequency.

FIG. 6 depicts optical microscope images showing changes in crystal habit for calcium oxalate crystals in a control solution and solutions including a linear polyphosphate material Na₅P₃O₁₀ at concentrations of 5 μM and 15 μM.

### DETAILED DESCRIPTION

This disclosure is related to new compounds and methods utilizing these new compounds that may act to reduce the incidence and/or reoccurrence of kidney stones and/or other pathological calcification symptoms or conditions.

In particular, polyphosphate materials are described herein as inhibitors of calcium oxalate crystal nucleation and growth. More generally, disclosed polyphosphate materials can be used to slow the rate of calcium mineral growth. Examples of calcium minerals include, without limitation, calcium oxalate, calcium phosphate, and calcium carbonate.

Disclosed polyphosphate materials include polyphosphates and polyphosphate derivatives that can be used in therapeutic treatments to prevent or slow the incidence of the formation of minerals (biomineralization) which can occur in a patient with various diseases or conditions, for example, without limitation, kidney stones, hypercalciuria, atherosclerosis (calcified plaque), and gout. Here, a “patient” is understood to encompass all mammals including humans. “Therapy” and “therapeutic treatment,” as used herein, encompass administering a compound to a patient for the purposes of curing a disease condition, ameliorating a disease condition, preventing a particular symptom of a disease condition, ameliorating a particular symptom of a disease condition, reducing the risk of the incidence or recurrence of a disease condition, or reducing the incidence, recurrence, or severity of a particular symptom of a disease condition.

Polyphosphate materials can be used in combination with other compounds for combination therapies to cure, ameliorate, or prevent conditions, symptoms, or diseases related to pathological calcification. The phosphate materials may be mixed with or into a pharmaceutically acceptable carrier. Acceptable carriers depend on intended route of administration. The administered composition may also include other active ingredients, adjuvants, and/or excipients.

### Polyphosphates

Polyphosphates are rich in negatively charged functional groups that interact with free calcium (Ca²⁺) ions in solution (via complexation) and/or with calcium at the surface of crystals (such as calcium oxalate monohydrate). The interaction between polyphosphate and calcium materials may function to inhibit calcium-compound crystallization.

Polyphosphates are anionic molecules consisting of multiple phosphate functional groups. In physiological environments (e.g., in vivo), the phosphate functional groups can exhibit a range of disassociated states according to the acid/base chemistry of the environment and the disassociation constants (pKa values) of the functional groups in the molecule. Polyphosphates molecules are generally water soluble. In an aqueous environment, the polyphosphate molecules can complex with other species in solution, such as ions, small molecules with ionic character, or larger molecules having at least portions with ionic character. In solid state, polyphosphates may be present as salts.

Polyphosphates can be conceptually grouped in to three different categories according to basic structure types: linear polyphosphates, cyclic polyphosphates (also referred to as “metaphosphates”), and branched polyphosphates (also referred to as “ultra-phosphates”). Linear polyphosphates include three or more phosphate groups connected in series. Cyclic phosphates include three or more phosphate groups connected in a ring structure. Branched phosphates include four or more phosphate groups or those in which at least three groups are directly attached to the fourth group. While the upper bound on the number of phosphate groups in a polyphosphate is not necessarily limited, the biocompatibility and/or aqueous solubility may eventually decrease for very large molecules. In some examples, it may be beneficial from either the standpoint of biocompatibility and/or crystallization inhibition effect for a polyphosphate molecule to include less than 20 phosphate groups, for example, 3 to 6 phosphate groups.

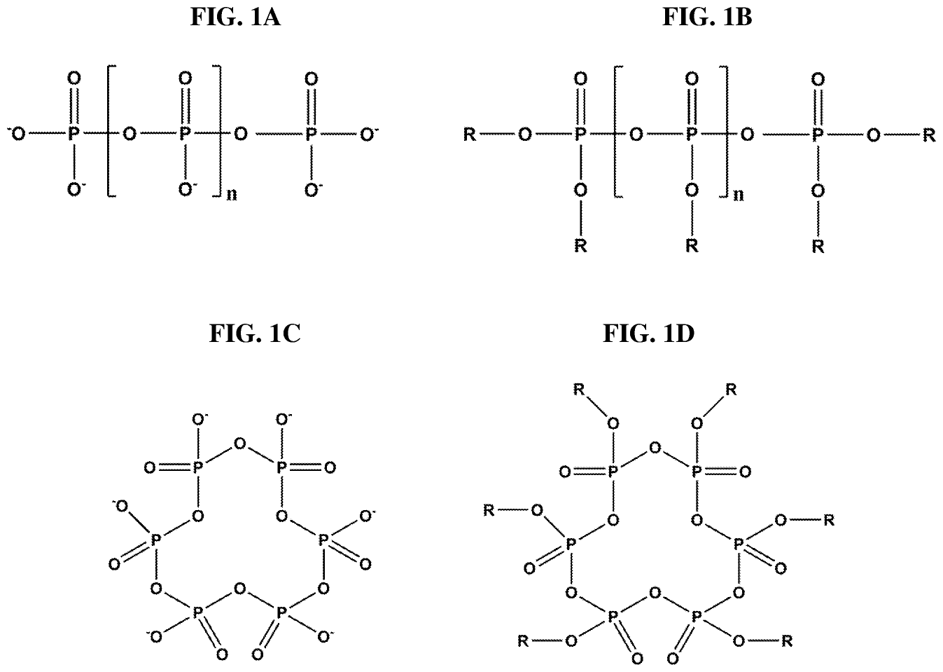

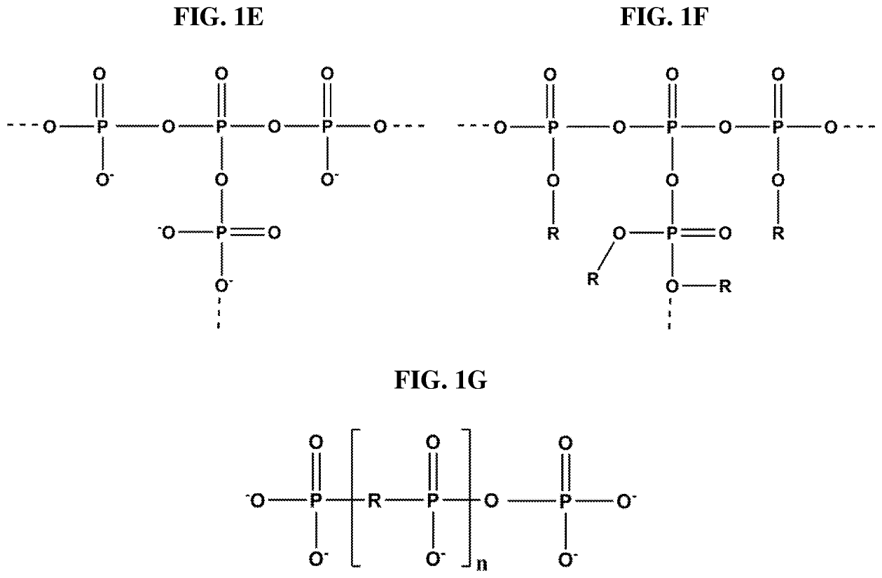

FIGS. 1A-1G depict structures of various types of polyphosphate materials. FIG. 1A depicts a linear polyphosphate and FIG. 1B depicts a functionalized/derivative form of the linear phosphate including “R” groups. FIG. 1C depicts a hexametaphosphate and FIG. 1D depicts a functionalized/derivative form of a hexametaphosphate with “R” groups. FIG. 1E depicts a branched polyphosphate and FIG. 1F depicts a functionalized/derivative form of a branched polyphosphate, with “R” groups. Branched polyphosphate may also be referred to as “ultraphosphate.” FIG. 1G depicts a functionalized/derivative form of a linear polyphosphate including an “R” group in the backbone.

In general, the “R” groups that can be used to derivatize or functionalize the polyphosphate materials of the present disclosure, including those in FIGS. 1B, 1D, 1F, and 1G can be any suitable substituent group. Each “R” group may be different from the other “R” groups in the same compound. That is, conceptually at least, each “R” group depicted in the functionalized/derivative forms may be independently selected even though, in practice, synthetic compatibility and site selectivity may have to be considered in selecting different “R” groups within the same molecule.

The examples of functional groups or “R” groups that can be used to derivatize or functionalize polyphosphates in preferred embodiments described herein include, without limitation, acyl groups, alkyl groups, cycloalkyl groups, cycloheteroalkyl groups, aryl groups, arylalkyl groups, acylamino groups, acyloxy groups, alkoxy groups, alkoxycarbonylamino groups, substituted alkenyl groups, alkenyl groups, alkylene groups, alkenylene groups, alkynyl groups, alkanoyl groups, fused aryl groups, alkaryl groups, arylamino groups, alkoxyamino groups, alkoxycarbonyl groups, alkylarylamino groups, alkylsulfinyl groups, alkylthio groups, amino groups, aminocarbonyl groups, aminocarbonylamino groups, arylalkyloxy groups, aryloxycarbonyl groups, arylsulfonyl groups, azido groups, bicycloaryl groups, bicycloheteroaryl groups, carbamoyl groups, carbonyl groups, carboxyamino groups, cycloalkoxy groups, cycloalkenyl groups, fused cycloalkenyl groups, cyanato groups, cyano groups, dialkylamino groups, halo groups, ethynyl groups, ethenyl groups, hydroxyl groups, nitro groups, heteroaryl groups, dihydroxyphosphoryl groups, aminohydroxyphosphoryl groups, thioalkoxy groups, sulfanyl groups, sulfonyl groups, sulfone groups, thioaryloxy groups, thioketo groups, thiol groups, and amino acid groups.

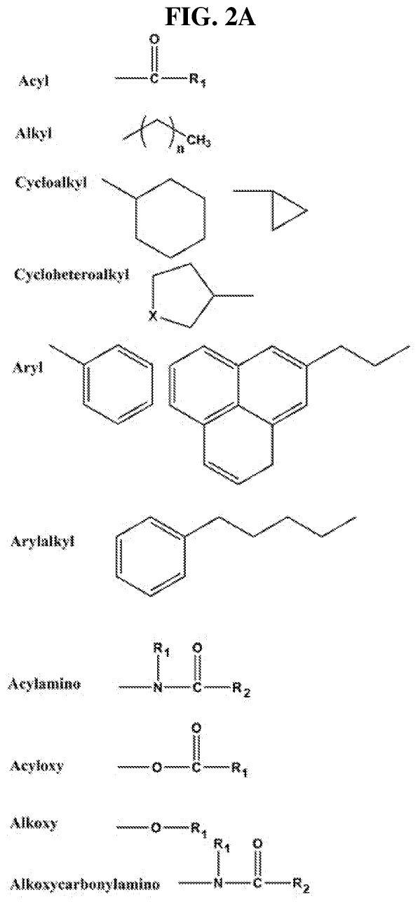

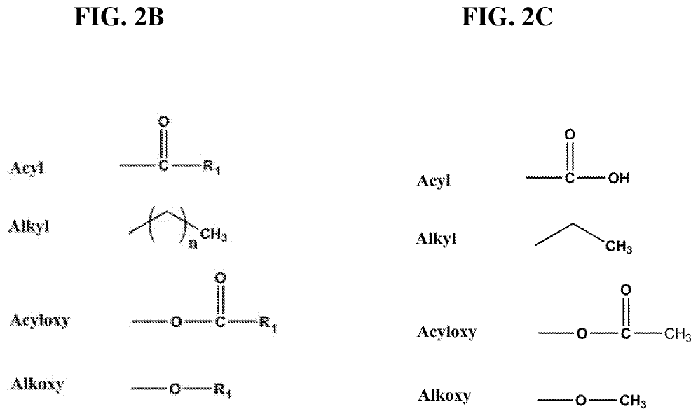

FIG. 2A depicts several possible functional groups that can be used as and/or incorporated in “R” groups on polyphosphates described herein in accordance with preferred embodiments, including those in FIGS. 1B, 1D, 1F, and 1G. FIG. 2A is not an exhaustive listing of possible functional groups. In FIG. 2, “n” indicates a repeating unit, “x” indicates a heteroatom (i.e., not carbon), and “R₁” and “R₂” are additional suitable functional groups. “R₁” and “R₂” may be independently selected when multiple “R₁” or “R₂” groups are present in a depicted group in FIG. 2A. FIG. 2B shows exemplary “R” groups having “R1” substituents, and FIG. 2C shows additional exemplary forms of “R” groups in which R₁ may be OH or CH₃. With regard to FIG. 1G, it is noted that the “R” group is in the main backbone of the phosphate material and thus the “R” group must have two bonds, which are not depicted in FIG. 2A-2C. Suitable adjustments to the “R” groups illustrated in FIG. 2A-2C can be made to address this, such as by using R₁ or R₂ groups that are CH₂ rather than CH₃.

This disclosure includes these various polyphosphates and polyphosphate derivatives and their corresponding salts, solvates, co-crystals, prodrugs, isomers, tautomers, and isotopic variants. For example, while the polyphosphates in FIGS. 1A-1F are depicted as unbound polyanions, these materials may be prepared as salts with corresponding cations, such as sodium or potassium or other alkali metals, or in solvated form in which surrounding solvent molecules compensate for the anionic character of the polyphosphate materials.

As one example, a linear tripolyphosphate (LTPP) having the following structure is disclosed:

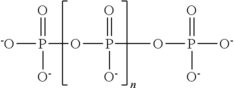

wherein n is 1 or more, in certain preferred embodiments n is between 1 and 8, and in additional preferred embodiments n is 1. Additional preferred embodiments include the salt form of the LTPP, such as Na₅P₃O₁₀.

In additional examples, a LTPP in accordance with preferred embodiments may have the following structure:

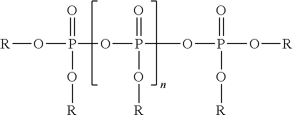

wherein n is 1 or more, in certain preferred embodiments n is between 1 and 20, and in additional preferred embodiments n is 1. The R groups may independently be any suitable R group identified above or in FIGS. 2A-2C. In further preferred embodiments R is independently selected from acyl, alkyl, acyloxy, and alkoxy groups.

As another example, in preferred embodiments a hexametaphosphate (HMP) having the following structure is disclosed:

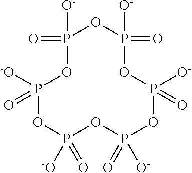

Additional preferred embodiments include the salt form of the HMP, such as (NaPO₃)₆.

As another example, a hexametaphosphate (HMP) having the following structure is disclosed:

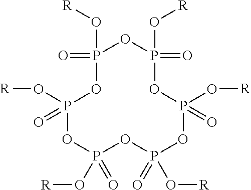

The R groups may independently be any suitable R group identified above or in FIGS. 2A-2C. In further preferred embodiments R is independently selected from carbonyl groups such as carboxylic acids.

In additional examples, a LTPP in accordance with preferred embodiments may have the following structure:

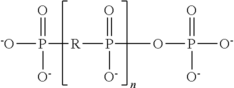

wherein n is 1 or more, in certain preferred embodiments n is between 1 and 20, and in additional preferred embodiments n is 4. The R groups may independently be any suitable R group identified above or in FIGS. 2A-2C, with accommodations made to address the need for two bonds to the main backbone of the structure. In further preferred embodiments R is independently selected from acyl, alkyl, acyloxy, and alkoxy groups.

In additional examples, a branched polyphosphate in accordance with preferred embodiments may have the following structure:

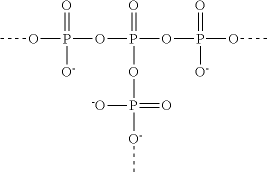

In additional examples, a branched polyphosphate in accordance with preferred embodiments may have the following structure:

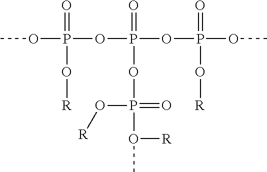

The R groups may independently be any suitable R group identified above or in FIGS. 2A-2C. In further preferred embodiments R is independently selected from acyl, alkyl, acyloxy, and alkoxy groups.

### Administration and Formulation

Disclosed polyphosphate materials may be administered to a patient by any known mechanism for drug delivery, such as, without limitation, oral, transdermal, and/or intravenous. Administration to the patient may be enteral or parenteral.

The polyphosphate materials may be included in, for example, capsules, tablets, pills, powders, or grains. These formulations may include, for example, starch, sucrose, lactose, talc, gelatin, sodium alginate, and polyvinyl alcohol. The polyphosphate materials may be included in, for example, syrups, elixirs, oil-in-water emulsions, water-in-oil emulsions, aqueous solutions, non-aqueous solutions, aqueous suspensions, or non-aqueous suspensions. The polyphosphate materials may be included in creams, gels, and ointments. The polyphosphate materials may be formulated for extended release.

Pharmaceutically acceptable carriers are materials that permit or allow the active ingredient(s) of a composition to be administered to a patient by at least one acceptable route. Here, the active ingredient would include polyphosphate materials. The pharmaceutically acceptable carrier is preferably safe for patient intake and compatible with the active ingredient. Depending on the intended administration route, the carrier may be a solid, a liquid, or a gas at an expected temperature for administration and/or storage, for example, approximately room temperature (25° C.).

The administered composition including the polyphosphate material may further include other active compounds intended to dissolve calcium oxalate crystals, inhibit calcium oxalate crystallization, or complex calcium ions. Buffers, diluents, stabilizers, flavorings, emulsifiers, suspending agents, binders, preservatives, and/or thickening agents and the like may also be incorporated when considered desirable or necessary. Routes of administration include, but are not limited to oral, dermal, inhalation, injection, and intravenous.

### Demonstration Responses

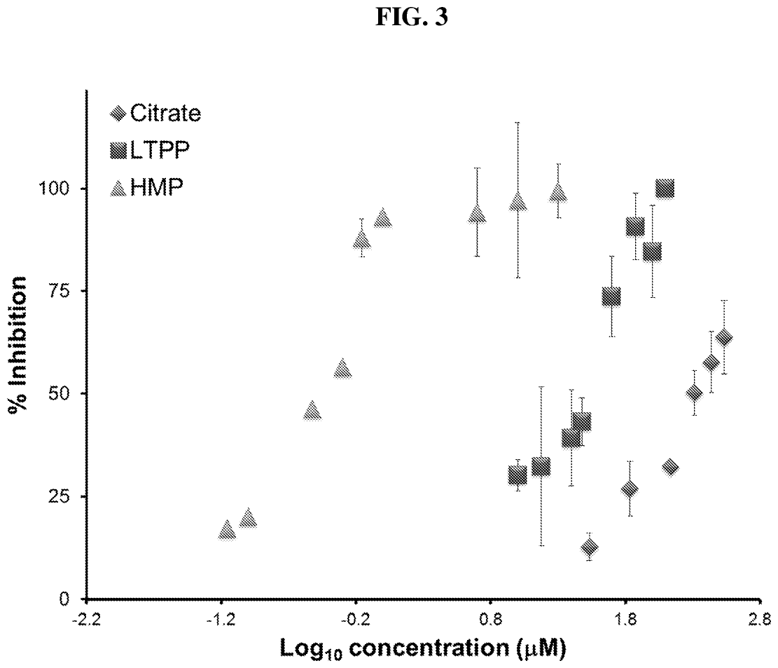

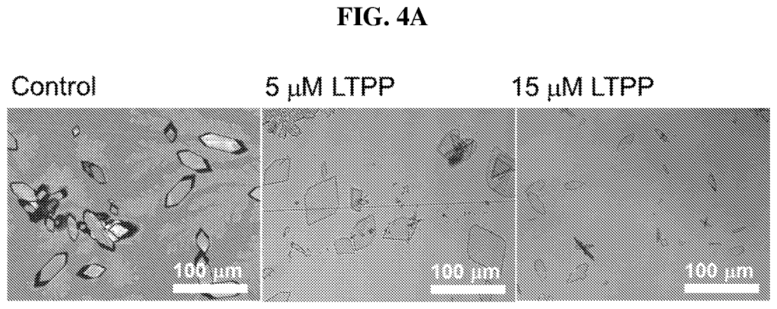

FIG. 3 depicts experimental results obtained using an example of a LTPP compound (Na₅P₃O₁₀) and an example of a HMP compound ((NaPO₃)₆) to inhibit crystallization of calcium monohydrate (COM) from aqueous solution. Results for a citrate compound (Na₃C₆H₅O₇), which is commonly used in the treatment of kidney stones, is presented for purposes of comparison. In FIG. 3, the x-axis represents a measure of concentration (micromolar (μM) concentration on a logarithmic scale) of the inhibitor present in the solution. The y-axis represents percent inhibition (% reduction in the rate of crystallization) of COM crystallization as compared to a control solution having no inhibitor compounds therein. The effect of the inhibitors on COM growth was estimated using ISE (ion selective electrode) measurements. This measurement technique enables quantification of the extent of inhibition on calcium crystal growth as a function of inhibitor concentration. More specifically, ISE measures free calcium concentration in solution as a function of time to measure the rate of crystallization in the absence and presence of inhibitor. This technique can be used to measure the effectiveness of the polyphosphates and polyphosphate derivatives at inhibiting or modifying calcium crystal growth. The values of “% inhibition” that were obtained by this technique, and presented in FIG. 3 for the different combinations of inhibitor type and concentration, reflect rates of crystallization that have been normalized with respect to the rate of crystallization for a control solution (e.g., inhibitor concentration of zero). The % inhibition refers to the averages of at least 3 separate experiments. The solutions tested were aqueous solutions of calcium chloride (CaCl₂)), sodium oxalate (Na₂C₂O₄), and sodium chloride (NaCl). The tested solutions further included the inhibitor/modifier compound at the stated concentration level.

While each inhibitor compound provides some inhibition effect, the HMP compound demonstrated approximately 100% inhibition at lower inhibitor concentrations than the other compounds depicted, including citrate, the current leading therapy. The HMP compound achieved approximately 100% inhibition at a concentration of less than about 1 μM. The LTPP compound demonstrated inhibition of 30-100% in a concentration range of about 5 μM to about 125 μM. The citrate compound appears to inhibit crystallization only up to about 60% even when much higher solution concentrations are used. Particularly, FIG. 3 shows the citrate compound achieves only about 60% inhibition at a concentration of around 320 μM.

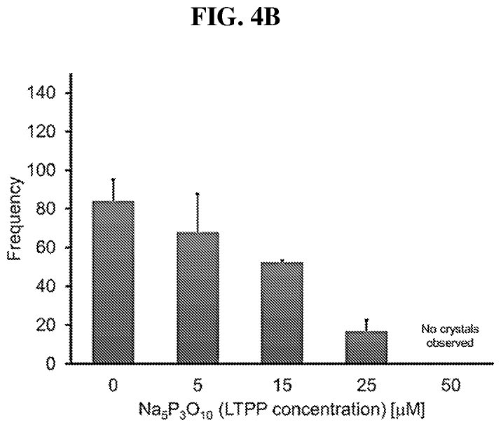

FIG. 4A depicts optical microscope images showing results of calcium oxalate crystallization from a control solution and solutions including LTPP Na₅P₃O₁₀ at concentrations of 5 μM and 15 μM. FIG. 4B depicts effects of Na₅P₃O₁₀ concentrations on calcium oxalate crystal frequency. Only very small crystals were observed as being formed from the LTPP containing solution at the 15 μM concentration level. Crystals formed from the control solution were approximately 25-50 μm in size as measured along the c-axis, which is the longest dimension. As shown in FIG. 4B, higher LTPP solution concentrations resulted in no definitive crystals being observed.

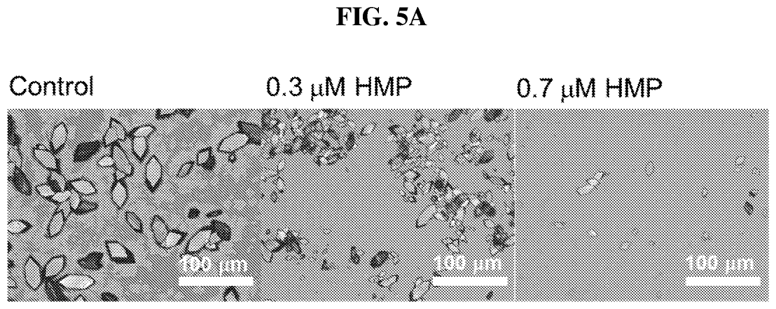

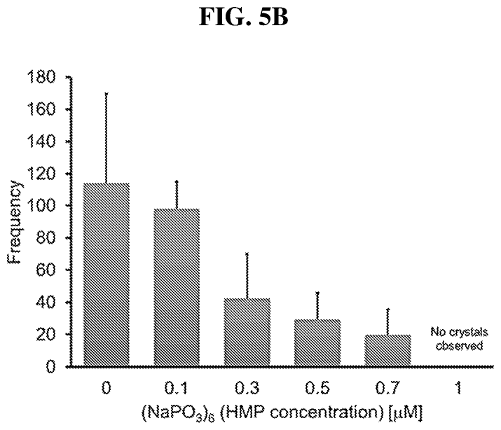

FIG. 5A depicts optical microscope images showing results of calcium oxalate crystallization from a control solution and solutions including HMP (NaPO₃)₆ at concentrations of 0.3 μM and 0.7 μM. FIG. 5B depicts effects of (NaPO₃)₆ concentrations on calcium oxalate crystal frequency. Very small crystals were observed as being formed from the HMP containing solution at the 0.7 μM concentration level. Crystals formed from the control solution were approximately 25-50 μm in size as measured along the c-axis, which is the longest dimension. As shown in FIG. 5B, higher HMP solution concentrations resulted in no definitive crystals being observed.

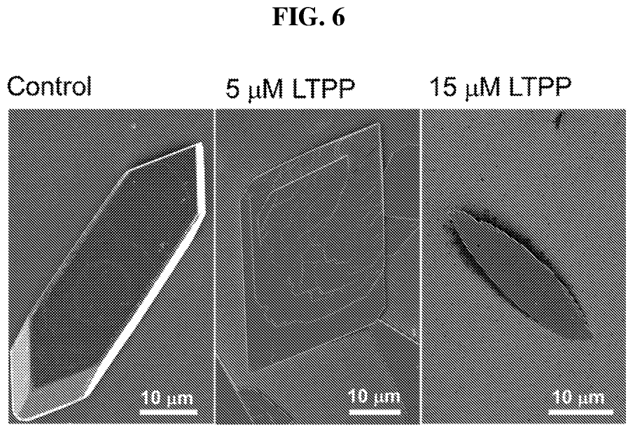

FIG. 6 depicts optical microscope images showing results of calcium oxalate crystallization during bulk studies at different concentrations of LTPP. The leftmost image is a representative control crystal. The middle image shows a crystal prepared with 5 μM LTPP. The rightmost image shows a crystal prepared with 15 μM LTPP. These results show that the morphology of the crystals change with increasing inhibitor concentration, which is indicative of their interaction with crystal surfaces during growth and is consistent with data in FIG. 3 showing that LTPP is an inhibitor of calcium oxalate crystallization.

Without being limited to any particular mechanism for polyphosphate inhibition of calcium oxalate or other minerals, crystal growth inhibition may occur through molecule adsorption onto the growing crystal surface. In such instances, the molecule may block or impede additional crystal material adding to existing surface(s) of the crystal. A polyphosphate (or a polyphosphate derivate) may include a number of possible sites available to adhere to the crystal surface. Furthermore, crystal growth may be hinder by polyphosphate molecules which act to at least temporarily bind or otherwise interact with free cations (e.g., Ca²⁺) in solution.

It is expected that polyphosphate materials that include derivatives or functionalized forms of the LTPP and HMP compounds tested herein would demonstrate similar or even improved effects because they would have enhanced interactions with the crystal material. It is further expected that LTPP compounds having a longer backbone than the LTPP compound tested herein would demonstrate similar or even improved effects because of the availability of multiple binding groups interacting with the crystal material.

While the foregoing is directed to embodiments of the inventions, other and further embodiments of the inventions may be devised without departing from the basic scope thereof.

It is contemplated that elements and features of one embodiment may be beneficially incorporated in other embodiments without further recitation. It is to be noted that the appended drawings illustrate only example embodiments presented for purposes of explanation of various aspects of the disclosure. These example embodiments are not to be considered limiting of the disclosure's scope.

1. A composition for inhibiting pathological calcification, comprising:
a linear tripolyphosphate (LTPP) material having a structure of 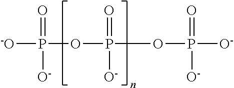
wherein n is from 1 to 8; and
a pharmaceutically acceptable carrier.

2. The composition of claim 1, wherein n is 1.

3. A method of treating or controlling pathological calcification in a patient, comprising administering the composition of claim 1 to the patient.

4. A composition for inhibiting pathological calcification, comprising:
a linear tripolyphosphate (LTPP) material having a structure of 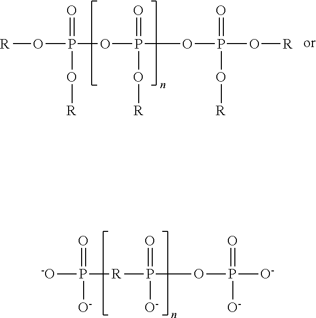
wherein n is from 1 to 20 and R is independently selected from acyl groups, alkyl groups, cycloalkyl groups, cycloheteroalkyl groups, aryl groups, arylalkyl groups, acylamino groups, acyloxy groups, alkoxy groups, alkoxycarbonylamino groups, substituted alkenyl groups, alkenyl groups, alkylene groups, alkenylene groups, alkynyl groups, alkanoyl groups, fused aryl groups, alkaryl groups, arylamino groups, alkoxyamino groups, alkoxycarbonyl groups, alkylarylamino groups, alkylsulfinyl groups, alkylthio groups, amino groups, aminocarbonyl groups, aminocarbonylamino groups, arylalkyloxy groups, aryloxycarbonyl groups, arylsulfonyl groups, azido groups, bicycloaryl groups, bicycloheteroaryl groups, carbamoyl groups, carbonyl groups, carboxyamino groups, cycloalkoxy groups, cycloalkenyl groups, fused cycloalkenyl groups, cyanato groups, cyano groups, dialkylamino groups, halo groups, ethynyl groups, ethenyl groups, hydroxyl groups, nitro groups, heteroaryl groups, dihydroxyphosphoryl groups, aminohydroxyphosphoryl groups, thioalkoxy groups, sulfanyl groups, sulfonyl groups, sulfone groups, thioaryloxy groups, thioketo groups, thiol groups, and amino acid groups; and
a pharmaceutically acceptable carrier.

5. The composition of claim 4, wherein n is from 1 to 8 and R is independently selected from acyl, alkyl, acyloxy, and alkoxy groups.

6. A method of treating or controlling pathological calcification in a patient, comprising administering the composition of claim 4 to the patient.

7. A composition for inhibiting pathological calcification, comprising:
a hexametaphosphate (HMP) material having a structure of 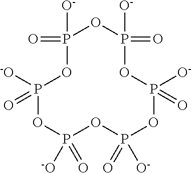
and a pharmaceutically acceptable carrier.

8. A method of treating or controlling pathological calcification in a patient, comprising administering the composition of claim 7 to the patient.

9. A composition for inhibiting pathological calcification, comprising:
a hexametaphosphate (HMP) material having a structure of 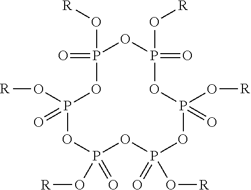
wherein R is independently selected from acyl groups, alkyl groups, cycloalkyl groups, cycloheteroalkyl groups, aryl groups, arylalkyl groups, acylamino groups, acyloxy groups, alkoxy groups, alkoxycarbonylamino groups, substituted alkenyl groups, alkenyl groups, alkylene groups, alkenylene groups, alkynyl groups, alkanoyl groups, fused aryl groups, alkaryl groups, arylamino groups, alkoxyamino groups, alkoxycarbonyl groups, alkylarylamino groups, alkylsulfinyl groups, alkylthio groups, amino groups, aminocarbonyl groups, aminocarbonylamino groups, arylalkyloxy groups, aryloxycarbonyl groups, arylsulfonyl groups, azido groups, bicycloaryl groups, bicycloheteroaryl groups, carbamoyl groups, carbonyl groups, carboxyamino groups, cycloalkoxy groups, cycloalkenyl groups, fused cycloalkenyl groups, cyanato groups, cyano groups, dialkylamino groups, halo groups, ethynyl groups, ethenyl groups, hydroxyl groups, nitro groups, heteroaryl groups, dihydroxyphosphoryl groups, aminohydroxyphosphoryl groups, thioalkoxy groups, sulfanyl groups, sulfonyl groups, sulfone groups, thioaryloxy groups, thioketo groups, thiol groups, and amino acid groups; and
a pharmaceutically acceptable carrier.

10. The composition of claim 9, wherein R is independently selected from carbonyl groups.

11. A method of treating or controlling pathological calcification in a patient, comprising administering the composition of claim 9 to the patient.
